(19) United States
(12) Patent Application Publication
(10) Pub. No.: US 2009/0030448 A1
(43) Pub. Date: Jan. 29, 2009

(54) SURGICAL FORCEPS, IN PARTICULAR AN OPHTHALMOLOGIC MICROSURGICAL FORCEPS

(76) Inventor: Jean-Marie Andre, Marseille (FR)

Correspondence Address: EGBERT LAW OFFICES, 412 MAIN STREET, 7TH FLOOR, HOUSTON, TX, 77002, US

(21) Appl. No.: 11/993,551

(22) PCT Filed: Jun. 22, 2005
(86) PCT No.: PCT/FR2005/001569

§ 371 Date: Jan. 29, 2008

(51) Int. Cl. A61B 17/00

(52) U.S. Cl. 606/205

(57) ABSTRACT

The invention relates to a surgical forceps, in particular an opthalmologic microsurgical forceps. The invention includes a gripping part moulded in one piece from a plastic material. The gripping part has two arms connected to one another at one end thereof. There is a clamping part formed by metal tips inserted into the distal ends of the arms during the moulding of the gripping part. The distal part of the forceps is equipped with centering devices having a cylindrical finger solidly connected to one of the branches of the forceps and oriented towards the other branch. The centering devices also have a transfixing hole provided in the other branch opposite the finger. According to the invention, the centering devices are provided in the end portions of the plastic arms, in which the proximal portions of the metal tips are inserted.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention concerns a surgical forceps, in particular, a single use opthalmologic microsurgical forceps, more specifically dedicated to cataract surgery.

More precisely, the invention refers to a disposable opthalmologic microsurgical forceps of the kind that has two branches that are linked to each other by one of their ends to form the proximal prehension part of the forceps and whose opposite ends are formed by pinch tips which can be brought together through elasticity and which form the active distal part of said forceps.

2. Description of Related Art Including Information Disclosed Under 37 CFR 1.97 and 37 CFR 1.98

Cataract operations are currently the most frequently performed surgical intervention in the world. This operation requires a number instruments and in particular microsurgical forceps of the aforementioned kind with extremely fine pinch tips, which, during their use, must be able to be rigorously brought together, one towards the other.

At present, these high precision instruments are most often manufactured by skilled manual labor, which makes them very costly.

Such forceps are described for instance in documents French Patent Nos. FR-2.644.689 and FR-2.491.325, the latter revealing the reasons for the necessarily very costly nature of these instruments.

The high cost of manufacture has been a major obstacle to making these instruments available as ‘disposables’ whereas their non-reuse would be desirable from both a sanitary and an economic standpoint. In effect, the sterilization and/or obligatory decontamination of reusable surgical instruments and their conservation in an aseptic condition, between two successive utilizations, require sizable investments in qualified personnel, in equipment and in working hours. On the other hand, these instruments become unusable as soon as their tips are just a little bit deformed or dulled and no longer present the indispensable operating precision.

In order to reduce the cost of these instruments so as to make them single-use items, it must be possible to mechanize their manufacture.

Document WO-03/04509, describes a disposable surgical forceps that has been made entirely by compression moulding of a thermoplastic material presenting itself in the form of a single monobloc piece.

The idea to produce microsurgical ocular forceps in a thermoformed plastic material may straight off appear to be very inviting, because of the economic aspect of this mode of production. The practitioners have been able to note, however, that while this material may be suitable for the manufacture of the sleeves or proximal parts of the branches of the instruments, it does not present the indispensable physical characteristics of finesse and hardness that are proper to metal, in the active distal part (the pinch tips of the branches) of said instruments.

To remedy the aforementioned drawbacks of surgical instruments made entirely of metal or of surgical instruments made of plastic material, it has already been proposed to make certain ones of these instruments with an active metallic part compound-filled by a plastic material injected under pressure (for example: US-2003/0109858, EP-1.262.157, WO-98/22035).

Document WO-98/22035 describes very summarily a type of disposable surgical forceps featuring two branches with metallic tips formed by compound-filled inserts.

Because of such a mode of manufacture it is possible to manufacture surgical instruments more economically allowing their single use.

However, the suppleness of plastic materials that can be used for the manufacture of microsurgical ocular forceps raises issues which the present invention proposes to resolve.

The biggest of these problems resides in the observation of the fact that if, for all-metal forceps, the rigidity of the metal permits a perfect alignment of the distal ends formed by the pinch tips of the branches of the forceps during their approach to each other, without there being any need for a particular centering device, the suppleness of the sleeves or proximal parts of said branches when they are made of plastic material is not capable of guaranteeing such a perfect alignment during their use.

For instance, in the document WO-98/22035, no provision has been made for a rigorous alignment of the pinch ends, so that the microsurgical forceps described in this document is not usable for microsurgical opthalmologic operations.

Document U.S. Pat. No. 4,041,952 on the other hand, describes a forceps for electrocoagulation the intended use and function of which are not the same as those of the microsurgical opthalmologic forceps that can be used for operations under an operating microscope in accordance with the invention. Also, according to modes of manufacture shown, the prehension part of the forceps is not moulded from a single plastic piece. According to the mode of manufacture of FIG. 2, the prehension part of the forceps comprises two arms that are separated by a spacer piece and held together by a sleeve; thus, there are four separately moulded pieces. Whereas, according to the mode of manufacture of FIG. 15, the prehension part includes a part in the shape of a fork which has been obtained by a first moulding operation and which includes two arms with grooves in which the electrical components of the forceps are located which are then enclosed in said grooves in a second moulding operation. In the first case, the non-simultaneous fastening of the metallic electrocoagulation inserts by means of a riveting process does not guarantee a rigorous reciprocal positioning of said inserts, nor of the centering devices. In the second case, this rigorous positioning is not any more guaranteed because of the second moulding operation which may disturb the positioning obtained by the first moulding.

### BRIEF SUMMARY OF THE INVENTION

According to the invention, the problem described above has been solved by a composite surgical forceps of the kind that comprises on the one hand a prehension part that is moulded from a single plastic piece and consisting of two arms interconnected by one of their ends in order to form the proximal end of the forceps and, on the other hand, a pinching part formed by metallic tips inserted during the moulding of said prehension part in the distal ends of said arms, so as to form the two branches of said forceps. The active distal part of this forceps is equipped with centering devices comprising on the one hand a male part, preferably consisting of a stud or finger in a cylindrical shape that is integral with one of the branches of the forceps and oriented towards the other branch and, on the other hand, a female part consisting of a transfixing orifice made in said other branch, facing said male part. These centering devices are provided in the end portions of the sleeves made of plastic material in which the proximal portions of the metallic pinch tips are implanted, so that said centering orifice transfixes or crosses not only the plastic material but also the part of the metallic tip inserted in said plastic material.

Because of this disposition, the active ends of the metallic pinch tips of the forceps can be brought together with great precision until they touch each other.

The orifice transfixing both the plastic material and the metallic part allows locating the centering devices as low as possible on the branches of the forceps and therefore as close as possible to the pinch tips of the forceps.

On the other hand, providing this transfixing orifice makes it possible to obtain the two arms of the forceps in one single moulding operation.

According to a preferred mode of manufacture the forceps is also provided with precentering devices including on the one hand, a male part, for example consisting of a peg that is integral with one of the branches of the forceps and oriented towards the other branch, and, on the other hand, a female part consisting of a transfixing orifice presented by the other branch, opposite said male part.

These precentering devices are located at a short distance (for example at a distance of 11.1 mm) from the previously described centering devices, in the direction of the proximal end of the forceps.

Advantageously the precentering peg and orifice present an oblong shape the large axis of which is oriented perpendicular to the axis of the branch.

The precentering peg and orifice are of a larger volume than that of the centering stud and orifice.

When the forceps branches come together, the precentering devices are the first to start acting thus allowing a sufficient leveling of said branches for the centering devices to then be perfectly able to fulfill their function.

Another problem encountered with the use of composite surgical forceps of the aforementioned kind, because of the suppleness of the plastic material constituting the forceps arms, is that it happens that when the active ends of the metal tips are pressed one against the other, the pressure exerted upon said arms by the practitioner's fingers can be greater than necessary. This results in a lever effect on a fulcrum located between the pressure area of the fingers and said active ends (for example consisting of the centering device, or even by the metallic platform of said tips) leading to a slight spreading of the latter, i.e., to a result that is exactly opposite to the pinching effect that is being sought.

According to another characteristic layout of the invention, this problem is solved by providing, on the internal face of at least one of the two plastic arms and at least in the prehension zone of the forceps, one or several stops oriented towards the internal face of the other arm.

According to a preferred mode of execution cooperating stops are disposed opposite each other on the internal face of each plastic sleeve of the forceps.

These stops effectively prevent an undesirable coming together of the sleeves of the forceps beyond their movement allowing the pinch tips of said forceps to come into contact with each other, even in the event of excessive pressure applied by the practitioner's fingers during the operation. On the other hand, providing a number of spaced cooperating stops spread over the prehension zone of the forceps contributes to the proper reciprocal positioning of the arms of the latter, in the pinching position.

### BRIEF DESCRIPTION OF THE SEVERAL VIEWS OF THE DRAWINGS

The above aims, characteristics and advantages and still others, will become clearer in the following description and the attached drawings.

FIG. 1 is a perspective view of an example of production of a microsurgical ocular forceps in accordance with the invention.

FIG. 2 is a perspective view and at a larger scale of the distal part of this forceps.

FIG. 3 is an elevation and front view of the forceps.

FIGS. 4 and 5 are side elevation views showing the lateral opposing sides of the forceps.

FIG. 6 is a longitudinal sectional view of the forceps shown in its resting position.

FIG. 7 is a longitudinal sectional view showing the coming together of the active tips of the forceps in the pinching position.

FIGS. 8 and 9 are sectional views, and at a larger scale, along lines 8-8 and 9-9 respectively of FIG. 3.

FIGS. 10 and 11 are partial elevation views showing the internal faces of the distal parts of the two branches of the forceps.

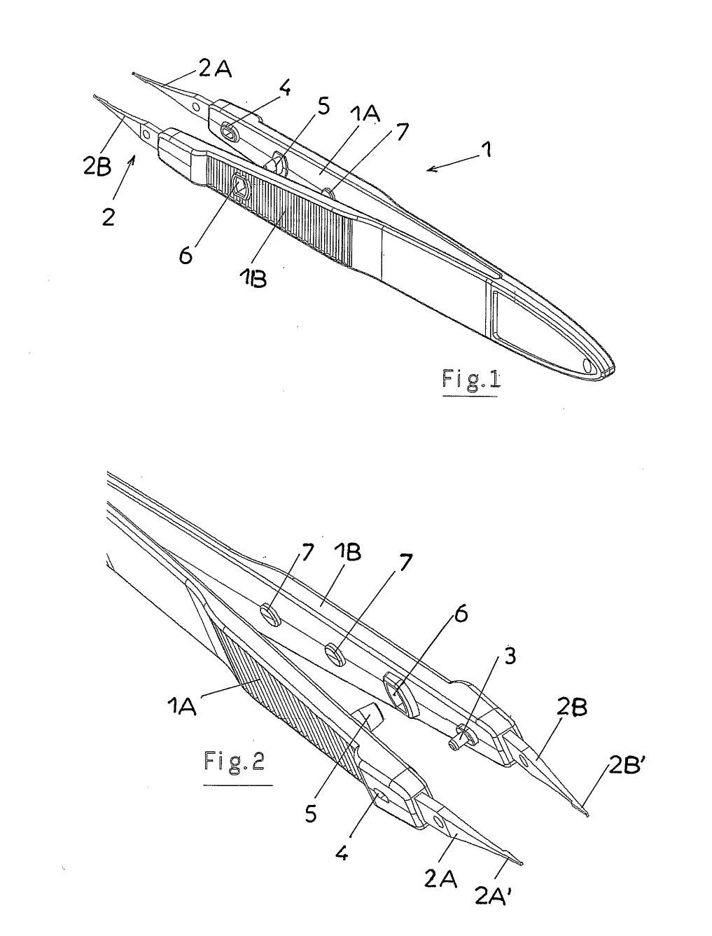

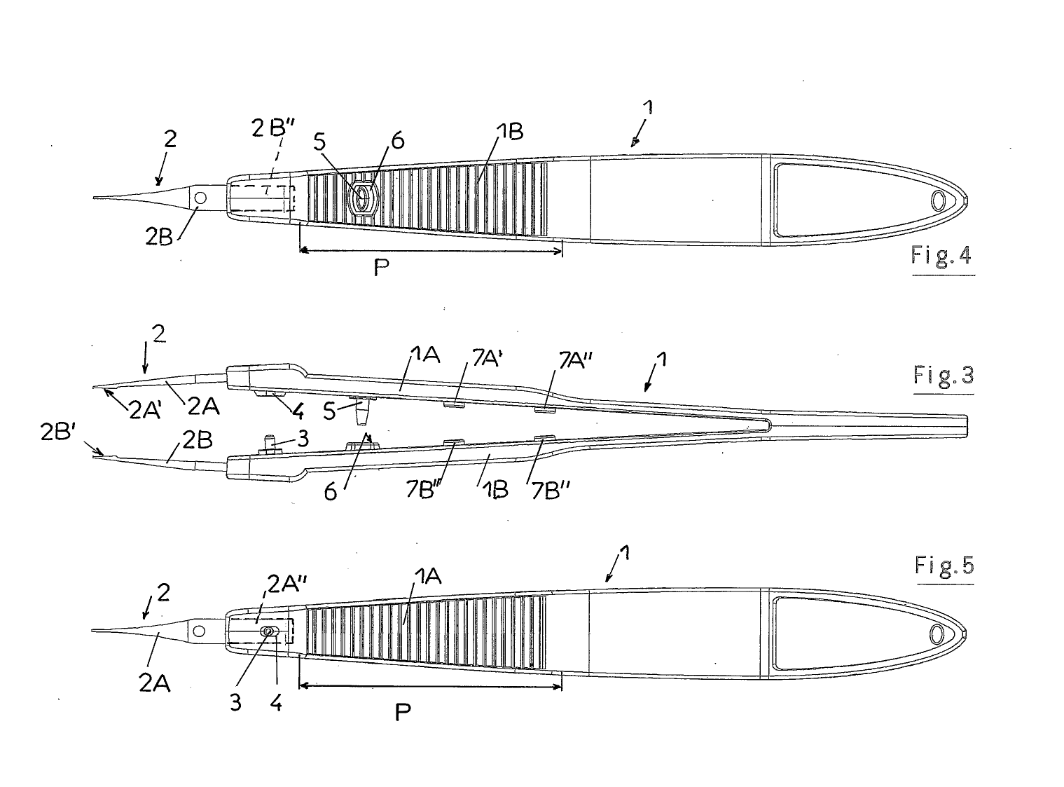

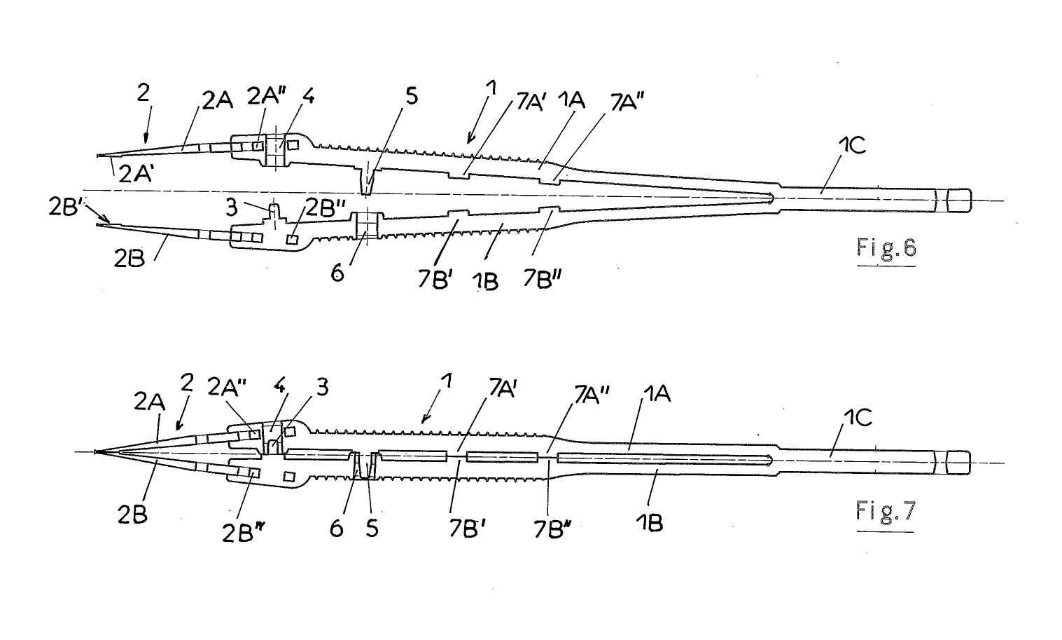

### DETAILED DESCRIPTION OF THE INVENTION

Reference is made to said drawings in order to describe an advantageous, although non-limiting, example of manufacture of a surgical forceps according to the invention which, according to this example, consists of one of the microsurgical ocular forceps usable in the operating process of a cataract.

The forceps referred to by the invention are of the type that has two branches that are connected to each other by of their ends so as to form the proximal part of the forceps and the opposite ends of which consists of pinch tips that can be brought together through elasticity and which constitute the active distal part of said forceps.

The forceps according to the invention is a composite forceps the manual prehension part of which is made of rigid and flexible plastic material such as, for example, ZYLAR™ (registered trade name), Terpolymers (ABS), Polycarbonates (PC) and where the pinching part is made of hard and non-deteriorating metal such as stainless steel, titanium or other.

This composite forceps is manufactured as one single piece, by duplicate moulding of its metallic pinching parts by any plastic material featuring the required hardness and flexibility characteristics, by any suitable injection process under pressure.

In this way the manufacture of metallic pinching tips in a metal of the desired hardness can be mechanized and automated, and the forceps can be obtained in a single injection operation which allows for a very economical production of the latter and their designation for single use.

The forceps manufactured in this manner include on the one hand, a prehension portion 1 executed in plastic material and consisting of two arms 1A, 1B connected to each other by one of their ends, to constitute the proximal end of the forceps and extending from their joint by forming an acute angle, for example in the order from 6° to 7° and, on the other hand, a pinching portion 2, consisting of metallic tips 2A, 2B that are implanted in the distal ends of said arms.

The arms 1A, 1B can be brought together through elasticity on account of the flexibility of the material they are made of, so as to bring the active ends 2A′, 2B′ of the pinching tips 2A, 2B respectively, into contact with each other.

The distal portion of the forceps manufactured in this manner is provided with centering devices so the active ends 2A′, 2B′ of the pinching tips 2A, 2B during their movement towards each other can be guided.

According to a first characteristic layout of the invention, these centering devices include on the one hand a male part, preferably consisting of a pivot 3 of a cylindrical shape, formed of a single piece with one 1B of the arms of the forceps made of plastic material and oriented towards the other arm 1A of the forceps, and on the other hand, a female part, consisting of a transfixing orifice 4 of oblong shape, made in the arm 1A, opposite said stud or finger 3. This orifice presents an oblong shape the large axis of which is parallel to the axis A-A of the arm 1A provided with said orifice (FIG. 11).

Pivot 3 and orifice 4 are located in the end portions of the plastic arms in which are embedded the proximal portions of the metallic pinching tips 2B and 2A respectively, so that said centering orifice 4 transfixes or crosses not only the plastic material but also part 2A″ of the metallic pinching tip 2A inserted into said plastic material.

According to another very advantageous characteristic layout of the invention, the forceps is also provided with precentering devices including on the one hand a male part, preferable consisting of a peg 5 formed of a single piece with one of the arms of the forceps and oriented towards the other arm and, on the other hand, a female part consisting of a transfixing orifice 6 which presents the other arm opposite said male part.

These precentering devices 5, 6 are located at a short distance from the centering devices 3, 4, in the direction of the joint end of arms 1A, 1B of the forceps.

According to a preferred way of manufacture, the precentering peg 5 and the transfixing orifice 6 have an oblong shape, for example approximately oval, with the long axis being oriented perpendicular to the axes A-A and A′-A′ of the arms 1A and 1B equipped with said peg and orifice respectively (FIG. 10). On the other hand, the precentering peg 5 and orifice 6 are of larger volumes than those of the centering pivot 3 and orifice 4 respectively.

The disposition of the constituent male and female elements of the centering devices 3, 4 and of the precentering devices 5, 6 on the arms 1A and 1B is preferably inverted, i.e., the centering pivot 3 is positioned on the arm 1B, whereas the precentering peg 5 is positioned on the arm 1A.

According to another characteristic layout of the invention the internal face of at least one of the arms 1A, 1B made of plastic material is equipped, at least in the prehension or digital pressure P area of said arms, with one or preferably several stops 7, oriented towards the internal face of the other arm 1B, 1A so as to limit the amplitude of the movement of the arms that is necessary to bring the active ends 2A′, 2B′ of the pinching tips into contact with each other.

For example, the internal face of at least one of the arms may be provided with two spaced stops 7, consisting of cylindrical studs oriented towards the internal face of the other arm. Alternatively, the internal face of at least one of the arms could be equipped with a single stop of considerable length covering for example most of the length of the prehension area of the latter.

According to a preferred way of manufacture, cooperating stops 7A′, 7B′ and 7A″, 7B″ are placed opposite each other on the internal face of each plastic arm 1A, 1B, between the centering devices 5 or 6 and the joint ends of said arms.

These stops are so dimensioned that when a joining force is applied in the prehension area P of the arms 1A, 1B, the stops 7A′, 7A″ of the arm 1A are pushed against the stops 7B′, 7B″ of the arm 1B, as soon as the active ends 2A\2B′ of the contact tips come into contact with each other.

One understands that because of this stop system, no excessive pressure that would be capable of causing a decentering of the active ends 2A′, 2B′ could be communicated to these during the operation.

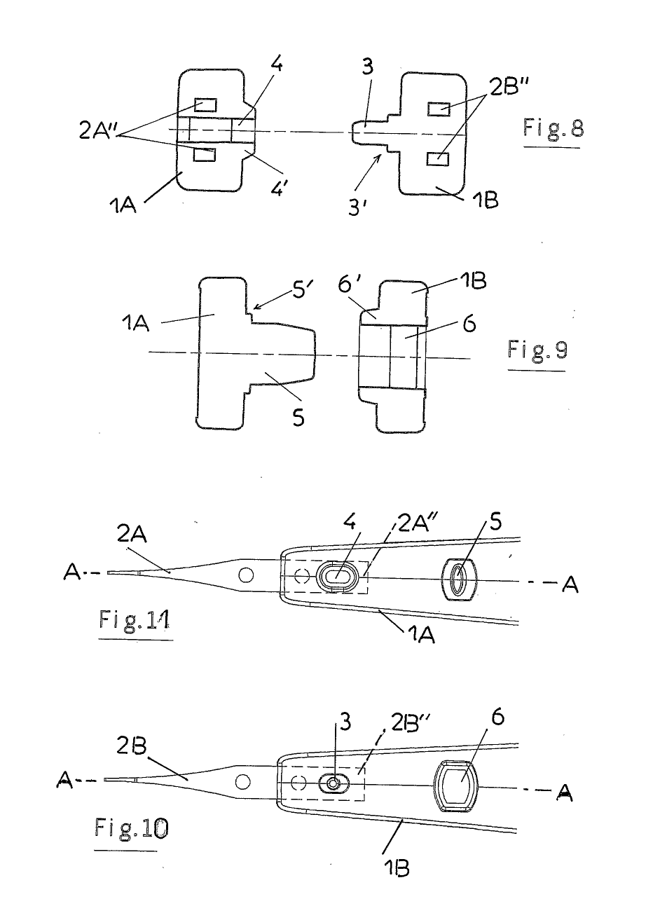

Interestingly, as is shown in FIGS. 8 and 9, the centering orifices 4 and/or the precentering ones 6 are flanged with a collar, 4′ and 6′ respectively, jutting out on the internal face of the arms 1A and 1B.

On the other hand, the centering finger 3 and/or the precentering stud 5 comprise, at their base, a circular shoulder 3′ and 5′ respectively.

These collars 4′ and 6′ and these circular shoulders 3′ and 5′ fulfill a function similar to that of the stops 7A′, 7B′, 7A″ and 7B″, when the precentering stud 5 and the centering finger 3 are engaged in the precentering orifices 6 and centering orifices 4.

While the branches 1A-2A and 1B-2B of the forceps come together, the stops 7A′, 7A″, the shoulder 5′ and the collar 4′ of the arm 1A come into contact with the stops 7B′, 7B″, the collar 6′ and the shoulder 3′ of the arm 1B, respectively, which stops the movement of said branches towards each other in the desired tightening position of the active ends 2A′, 2B1 of the pinching tips 2A, 2B while preventing any possibility of a too forceful pinching, whatever the force of the pressure applied by the practitioner might be.

The various stops 7A-7B′, 7A″-7B″, 5′-6′, 3′-4′, are spread over the entire length of the prehension area of the forceps so that there is no possibility of one of the branches tipping around a fixed support point, in the event of too much pressure on said branches.

It has been described and illustrated, for the presentation of the invention, a particular type of ocular microsurgical forceps that is used during the operating procedure for cataracts, but it is obvious that the invention is not limited to this type of forceps. It applies to all forcepses used in the performance of the aforementioned operation and in a general way to all microsurgical forcepses of comparable architecture for which there is the need for single-use instrumentation such as forcepses for plastic, vascular, restoring, dentistry and other microsurgery.

1. Opthalmologic microsurgical forceps, for single use, comprising:
a prehension part moulded in one piece of plastic material and being comprised of two arms, connected to each other by one end thereof and forming a proximal end; and
a pinching part being comprised of metallic tips inserted, during moulding of said prehension part, into a distal part of said two arms, forming two branches, said distal part being equipped with centering devices, each centering device comprising a male part formed by a stud of cylindrical shape integral to one of the branches and oriented towards the other branch, and a female part formed by a transfixing orifice made in said other branch, opposite said male part, said centering devices being provided in said ends said two arms at the proximal portions where the metallic pinching tips are inserted, said transfixing orifice crossing said plastic material and a part of the metallic tip mounted into said plastic material.

2. Opthalmologic microsurgical forceps, for single use, as per claim 1, wherein said transfixing orifice has an oblong shape and a longitudinal axis parallel to an axis of the arm provided with said transfixing orifice.

3. Opthalmologic microsurgical forceps, for single use, as per claim 1, further comprising:
precentering devices comprising: a male part formed by a peg integral with one of the branches and oriented towards the other branch, and a female part being comprised of a transfixing orifice presented on the other branch, opposite said male part, said precentering devices being located at a distance from said centering devices in a direction of the proximal end.

4. Opthalmologic microsurgical forceps, for single use, as per claim 3, wherein male and female elements of said centering devices and said precentering devices are inverted.

5. Opthalmologic microsurgical forceps, for single use, as per claim 3, wherein said peg and said transfixing orifice have an oblong shape and a longitudinal axis oriented perpendicular to axes of the arms equipped with said peg and said transfixing orifice, respectively.

6. Opthalmologic microsurgical forceps, for single use, as per claim 3, wherein said peg and said transfixing orifice have larger volumes than the centering pivot and orifice, respectively.

7. Opthalmologic microsurgical forceps, for single use, as per claim 1, wherein at least one of the plastic arms has an internal face equipped, at least in an area of prehension or of digital pressure of the arms, with one or several stops oriented in a direction of said internal face of the other arm.

8. Opthalmologic microsurgical forceps, for single use, as per claim 7, wherein said internal face of each plastic arm is equipped with spaced cooperating stops situated opposite each other, between the precentering devices and joint ends of said arms.

9. Opthalmologic microsurgical forceps, for single use, as per claim 7, wherein the centering finger and/or the precentering stud have a circular shoulder at a base thereof, the centering and/or precentering orifices being flanged by a collar, the shoulders and the collars coming into contact with each other in a pinching position, forming stops during contraction of the branches.

10. Opthalmologic microsurgical forceps, for single use, as per claim 7, wherein the stops are distributed over a length of the prehension area, restricting joining movement of the arms.
